(19) United States
(12) Patent Application Publication
(10) Pub. No.: US 2017/0216329 A1
(43) Pub. Date: Aug. 3, 2017

(54) Ceramide-Rubusoside Nanomicelles and Their Use in Cancer Therapy

(71) Applicants: Zhijun Liur, Baton Rouge, LA (US); Yong-Yu Liu, West Monore, LA (US)

(72) Inventors: Zhijun Liu, Baton Rouge, LA (US); Yong-Yu Liu, West Monroe, LA (US)

(73) Assignees: Board of Supervisors of Louisiana State University and Agricultural and Mechanical College, Baton Rouge, LA (US); Board of Supervisors for the University of Louisiana System, acting on behalf of the; University of Louisiana at Monroe, Monroe, LA (US)

(21) Appl. No.: 15/308,784

(22) PCT Filed: May 6, 2015
(86) PCT No.: PCT/US2015/029379

§ 371 Date: Nov. 3, 2016

Related U.S. Application Data
(60) Provisional application No. 61/989,709, filed on May 7, 2014.

(51) Int. Cl. A61K 31/704; A61K 47/26; A61K 45/06; A61K 9/107; A61K 31/164

(52) U.S. Cl. CPC A61K 31/704; A61K 9/1075; A61K 31/164; A61K 45/06; A61K 47/26

(57) ABSTRACT

Water-soluble, cell-permeable nanomicelles containing ceramides and a steviol glycoside, such as rubusoside, are disclosed. The ceramide-steviol glycoside complex has high water solubility, and can be used for treating cancers with p53 mutations. Preliminary results have shown that the Cer nanomicelles are effective in restoring p53 protein expression, and that they are functionally dominant over p53 mutants. The novel nanomicelles restore a wild-type phenotype, and have very low toxicity to noncancerous cells. The novel Cer nanomicelles may be used in treating p53-associated cancers.

The benefit of the 7 May 2014 filing date of U.S. provisional patent application Ser. No. 61/989,709 is claimed under 35 U.S.C. §119(e) in the United States, and is claimed under applicable treaties and conventions in all countries.

### TECHNICAL FIELD

This invention pertains to inhibiting cancers with p53 mutations, particularly to inhibiting such cancers using ceramide-steviol glycoside nanomicelles.

### BACKGROUND ART

The tumor suppressor protein p53 plays a key role in preventing tumorigenesis and cancer progression. The function of p53 is compromised in many cancers as a result of mutation. The frequency of p53 mutation varies among different cancer types. Mutations of p53 appear in some 50-70% of ovarian, colorectal, and head and neck cancers. The p53 mutants lose wild-type (wt) function. In addition, the p53 mutants often exert a dominant-negative regulation over any remaining wt p53 genes, and they can gain oncogenic functions that are independent of wt p53, promoting resistance to therapies and producing a poor prognosis. The p53 mutants are a target for improved cancer treatments.

As a transcription factor, p53 activates the expression of p21, Bax, Puma, Fas, and other p53-responsive genes. Effects of normal, wild-type p53 function include arresting cell growth, increasing autophagy, increasing apoptosis, and enhancing DNA repair. Trans-activation of p53 is sequence-specific, and is selective for the p53 DNA-binding domain (DBD). Most oncogenic p53 mutations occur in the DBD. These mutations inhibit DBD-binding to the promoters of p53-responsive genes. Because the p53 mutants have a dominant-negative activity, and because they exhibit oncogenic gain-of-function, prior approaches that have attempted to reactivate p53 function have had only limited success—merely reactivating wild type (wt) p53 function does not, in itself, reduce mutant-associated activity.

Tumor suppressor p53, which is encoded by the human gene TP53, functions primarily as tetrameric transcriptional factor. It regulates a large number of genes and microRNAs in response to a variety of cellular insults, including oncogene activation and DNA damage. Trans-activation by p53 protein (393 amino acids) involves five protein domains, including the transactivation (TA), proline-rich (PR), DNA-binding (DBD), tetramerization (Tet), and regulatory domains. In many tumor cells p53 function is compromised, usually the result of a somatic mutation. A mutation in TP53 occurs in approximately half of all human cancers. The frequency of TP53 mutations varies considerably among cancer types, ranging from ˜10% (e.g., hematopoietic malignancies) to ˜50-70% (e.g., ovarian, colorectal, and head and neck cancers). Most cancer-associated mutations in TP53 (about 65-80%) have been reported to be missense mutations within the DBD domain. Based on mutation frequencies seen in tumor cases, a number of “hotspots” have been identified, including R175, G245, R248, R249, 8273 and 8282. In addition to causing a loss of wt p53 activity, these mutations usually give the mutant protein (mutant p53) a dominant-negative activity over the remaining wt p53, through a mechanism that may involve hetero-oligomerization of mutant protein and wt protein. Moreover, p53 mutants tend to acquire oncogenic functions that are independent of wt p53. Additionally, both somatic and germline TP53 mutations are usually followed by loss of heterozygosity (LOH) during tumor progression, which suggests that selective forces allow the mutant allele to express mutant proteins that result in a mutant phenotype and loss of function of the remaining wt allele.

Prior attempts to restore wt p53 function in cancer treatments have focused on three main approaches: 1) replacement of wt p53 by gene therapy; 2) augmentation of wt p53 by inhibiting MDM2-mediated p53 protein degradation; or 3) reactivation of p53-mutant binding to DNA by altering the p53-mutant protein conformation. However, the dominant-negative activity and the oncogenic function of p53 mutants frequently interfere with these approaches. There are no prior reports of successfully restoring wt p53 expression dominance over mutant p53.

Gene therapy for p53 replacement delivered by adenoviral vectors has been attempted in some clinical trials. Although these studies provided proof-of-principle for p53 replacement, unfortunately it was observed that adenoviral p53 gene therapy did not result in significantly improved outcomes as compared to standard chemotherapy for ovarian cancers. Several factors have been suggested as limiting the efficacy of p53 gene therapy, including inefficient systemic delivery, non-specific immune responses, and the presence of p53 mutants in cancers. Another approach has been to employ small molecules (e.g., Nutlin, MI-219 and RITA) to interfere with p53-MDM2 protein interactions and thereby enhance wt p53 activity by slowing p53 degradation. Although this approach has shown some promising results against human tumor xenografts in preclinical models, the dominant-negative effects and oncogenic gain-of-function of p53 mutants can compromise the effectiveness of these compounds. One study found that introducing wt p53 into p53 missense-mutant cells was insufficient to overcome a malignant phenotype or drug resistance. Restoring wild-type function by modulating protein conformation has been tried in some studies. Compounds that directly target missense p53-mutants (e.g., PhiLan083, CP-31398, Ellipticine, PRIMA-1) have also been tested. However, because p53 protein binds to promoters a tetramer the efficacy of these compounds may be limited by the formation of hetero-tetramers of wt p53 with mutant p53, or homo-tetramers of mutant p53.

Relatively little work has been reported concerning attempts to restore normal p53 expression in cancer cells harboring p53-mutants. (Patwardhan et al., 2009) and (Liu et al., 2011) reported that suppressing GCS by MBO-asGCS restored wt p53 expression and p53-dependent apoptosis in p53-mutant ovarian cancer cells, including NCI/ADR-RES and OVCAR-8 (deletion codons 126-133, codon 126-132 in exon 5, respectively). The restored p53 activated the expression of p21, Puma, and Bax, and induced apoptosis in p53-mutant tumors.

Ceramides (Cers) are waxy lipid molecules comprising a sphingosine (long-chain amino diol) backbone, covalently bonded to a fatty acid residue via an amide linkage. Cers are found in many cell membranes. Mammalian Cers include more than 200 molecularly distinct species, with various saturated/unsaturated acyl chains. See Hannun, Y A., and Obeid, L. M. (2011). Many ceramides. J Biol Chem 286, 27855-27862; Merrill, A. H., Jr. (2011). Sphingolipid and glycosphingolipid metabolic pathways in the era of sphingolipidomics. Chem Rev 111, 6387-6422.

Cers can promote apoptosis. It has been proposed that Cers may have therapeutic efficacy. See, e.g., Hannun, Yusuf A., and Lina M. Obeid. “Principles of bioactive lipid signalling: lessons from sphingolipids.” Nature reviews Molecular cell biology 9.2 (2008): 139-150. However, Cers are highly hydrophobic, a property that has limited prior studies of Cer interactions with protein or RNA to regulate gene expression, or to modulate apoptosis in vivo. Their poor aqueous solubility has been a bottleneck both for mechanistic studies and for the development of exogenous Cer-based therapeutic agents.

Liu, Y. Y. (2011). Resuscitating wild-type p53 expression by disrupting ceramide glycosylation: a novel approach to target mutant p53 tumors. Cancer Res 71, 6295-6299 reported evidence suggesting that disrupting ceramide glycosylation could resuscitate wild-type p53 expression and p53-dependent apoptosis in mutant p53 tumors.

In Patwardhan et al. 2014 we and our collaborators reported experimental data showing that ceramide modulates pre-mRNA splicing to restore p53 expression. Endogenous long-carbon chain ceramide species (C₁₆- to C₂₄-ceramides) and exogenous C₆-ceramide can restore the function of wild-type p53 mRNA, of the phosphorylated p53 protein, and of p53-responsive proteins, including p21 and Bax, in ovarian cancer cells in vitro. Ceramide was supplied as a 5 μM solution in 5% FBS medium. Ovarian cancer cells predominantly have a deleted p53 exon-5 mutation. The restored p53 function sensitized the p53-mutant cancer cells to DNA damage-induced growth arrest and apoptosis. We reported evidence that ceramide activates protein phosphatase-1, and that the dephosphorylated serine/arginine-rich splicing-factor 1 (SRSF1) is then transported to the nucleus, where it promotes pre-mRNA splicing preferentially for wild-type p53 expression. Ceramide, through its effects on SRSF1, restored wild-type p53 expression preferentially over expression of the deletion-mutant, and promoted cancer cell apoptosis.

Cer is central to sphingolipid metabolism. Cer glycosylation catalyzed by GCS converts Cer to glucosylceramide. Disrupting Cer glycosylation can alter cellular levels of several sphingolipid molecules, including sphingosine and sphingosine 1 phosphate (S1P). Individual Cer molecular species are regulated by specific biochemical pathways in distinct subcellular compartments, and execute distinct functions. (Liu et al., 2011) described studies seeking to understand how suppression of Cer glycosylation affected restoration of wt p53 expression. Electrospray ionization tandem mass spectrometry (ESI-MS/MS) analysis showed that among 13 detectable Cer species, MBO-asGCS treatment significantly increased the C₁₆-, C₁₈-, C₂₀-, C₂₂-C₂₄-, C_{24:1}- and C₂₆-Cer species. Cellular C₁₈-Cer was increased threefold (0.023 vs. 0.073 pmol/μg protein), C₂₀-Cer increased by threefold, C₂₂-Cer by twofold and C₂₄-Cer by threefold (0.66 vs. 1.98 pmol/μg protein) respectively, compared to levels in cells that had been treated with a scrambled oligonucleotide control (MBO-SC). However, MBO-asGCS treatments did not significantly alter levels of other sphingolipids, including C₁₆-dihydroceramide, dihydrosphingosine, sphingosine, and S1P in NCI/ADR-RES cells, as compared to cells treated with MBO-SC or vehicle. MBO-asGCS treatment significantly increased wt p53 (phosphorylated-Ser15) by more than 8 times. Levels of p21 and Bax, which are p53-responsive genes, rose concomitantly in tandem with substantial rises in concentrations of endogenous Cer in the cytoplasm. Fumonisin B1 (FB1) treatment, which inhibits Cer synthase, has inhibited p53 restoration by MBO-asGCS. Exogenous C₆-Cer, but not C₆-dihydroceramide, restored p53 activity in a manner similar to that seen with MBO-asGCS treatment.

Cer modulates the restoration of wt p53 expression in missense mutant cancer cells. The primary alterations in most tumor-associated p53 mutations are missense mutations resulting in a single amino acid substitution, and most of these occur in the core DNA-binding domain (DBD). A mutation in codon 248 is among the most commonly observed in human cancers. OVCAR-3 human ovarian cancer cells carry the missense mutation R248G (Arg-*Gln) in exon 7. MBO-asGCS treatment significantly increased OVCAR-3 sensitivity to doxorubicin (Dox) by eightfold (EC₅₀, 0.09 vs. 0.75 μM). Furthermore, MBO-asGCS treatment dramatically increased the expression of p21, which is a p53-responsive gene that determines cell-cycle arrest. Isogenic SW48/TP53^{+/-} cells, in which the heterozygous missense mutation R248W in TP53 is knocked-in (Arg→Trp), provide an model for further mechanistic studies. Introduction of a heterozygous (HTZ) R248W mutation has been reported to cause SW48 human colon cancer cells to gain resistance to anticancer drugs. We have found that HTZ SW48/TP53^{+/-} and its parental SW48 (wt p53) cells were equally sensitive to Cers, and that both were more sensitive to C₁₈-Cer than C₆-Cer (data not shown).

Endogenous Ceramides are a family of related molecules, including fatty Cers from C₁₂ to C₃₆. Ceramide synthases 1-6 (CerS1-6), which are encoded by several genes, catalyze N-acylation of the sphingoid long-chain base, attaching acyl-CoAs with C₁₄-C₂₆ acyl chains to sphinganine to form C₁₄-C₂₆ dihydroceramides, which in turn are converted into C₁₄-₂₆-Cers by dihydroceramide desaturase in the mammalian endoplasmic reticulum. Each CerS has a high selectivity towards one or a few acyl CoA chain lengths in the N-acylation. Thus, the particular CerSs are responsible for the fatty acid compositions of the Cer in a cell: CerS1 catalyzes the production of C₁₈-Cer. CerS4 catalyzes the production of C_{18,20}-Cers. CerS5 catalyzes the production of C₁₆-Cer production. CerS6 catalyzes the production of C₁₄-Cer, C₁₆-Cer, and C₁₈-Cer. A Cer can be converted to a glycosphingolipid, catalyzed by glucosylceramide synthase (GCS); or it can be degraded by ceramidase.

Enhancing cellular Cer levels in cells has been reported to enhance the therapeutic response to chemotherapy and radiation therapy. Cer acts as a “second messenger” in regulating cell growth arrest, apoptosis, cell senescence, and autophagy. Conversely, resistance to chemotherapy has been associated with the loss of Cer generation, and with enhanced Cer glycosylation.

The native concentrations of Cers in vivo differ for different chain lengths. Some functions of Cers have been reported to be chain-length-dependent. For example, short chain Cers, such as C₆-Cer, have been reported to induce apoptosis and modulate gene regulation. See (Liu et al., 2011; Liu et al., 2008). (Adiseshaiah et al., 2013; Jiang et al., 2011; Stover et al., 2005; van Vlerken et al., 2008) reported studies employing nanoliposomes (PGE-DSPE) to deliver C₆-Cer in tumor xenograft models, and finding decreased tumor growth. (Liposomes are artificial vesicles made of components similar to those of cell membranes.)

Surfactants, molecules having both hydrophilic and hydrophobic groups, can associate with one another in polar solvents such as water to form dynamic aggregates known as “micelles.” A micelle typically takes roughly the shape of a sphere, a spheroid, an ellipsoid, or a rod, with the hydrophilic groups on the exterior and the hydrophobic groups on the interior. The interior provides, in effect, a hydrophobic liquid phase with solvation properties differing from those of the surrounding polar solvent. Although “traditional” surfactants typically have a long hydrocarbon tail covalently bonded to an ionic or polar moiety, such a configuration is not essential. Compounds with other structures can also act as surfactants, provided they possess a conformation with well-separated hydrophobic and hydrophilic domains. A “nanomicelles” is a micelle whose diameter is about 1000 nanometers or smaller.

The natural compound rubusoside, for example, can act as a surfactant. Rubusoside is a steviol glycoside, an intense sweetener, but one that is not actually found in Stevia. Rubusoside instead can be extracted from the Chinese plants Rubus suavissimus and Rubus chingii. In addition to its sweetening properties, rubusoside also has been reported to have solubilizing properties. See, e.g., published patent application US 2011/0033525; Zhang, Fang, Gar Yee Koh, Duane P. Jeansonne, Javoris Hollingsworth, Paul S. Russo, Graca Vicente, Rhett W. Stout, Zhijun Liu. 2011. A novel solubility-enhanced curcumin formulation showing stability and maintenance of anti-cancer activity. Journal of Pharmaceutical Sciences 100: 2778-2789; Zhang, Fang, Gar Yee Koh, Javoris Hollingsworth, Paul S. Russo, Rhett W. Stout, Zhijun Liu. 2012. Reformulation of etoposide with solubility-enhancing rubusoside. International Journal of Pharmaceutics 434: 453-459; and Zhijun Liu, Fang Zhang, Gar Yee Koh, Xin Dong, Javoris Hollingsworth, Jian Zhang, Paul S. Russo, Peiying Yang and Rhett W. Stout. 2015. Cytotoxic and antiangiogenic paclitaxel solubilized and permeation-enhanced by natural product nanoparticles. Anti-Cancer Drugs 26(2): 167-179.

Hannun, Y A., and Obeid, L. M. (2008). Principles of bioactive lipid signalling: lessons from sphingolipids. Nat Rev Mol Cell Biol 9, 139-150 provides a review of the role of sphingolipids such as ceramide as signalling and regulatory molecules.

Massiello, A., and Chalfant, C. E. (2006). SRp30a (ASF/SF2) regulates the alternative splicing of caspase-9 pre-mRNA and is required for ceramide-responsiveness. J Lipid Res 47, 892-897 reported evidence that ceramide modulates isoform protein expression.

There is an unfilled need for improved compositions and methods for treating cancers, particularly cancers with p53 mutations because such cancers are unfortunately so common.

### DISCLOSURE OF THE INVENTION

We have discovered water-soluble, cell-permeable nanomicelles containing ceramides and a steviol glycoside, such as rubusoside. The nanomicelles can made be made either with native Cers, having no chemical modifications, or with modified Cers. The ceramide-steviol glycoside complex has high water solubility, and can be used for treating cancers with p53 mutations. The nanomicelles may be administered through any route that is convenient for the practitioner and the patient, and they may even be administered orally without adversely affecting bioavailability and efficacy. Our preliminary results have shown that the Cer nanomicelles are effective in restoring p53 protein expression, and that they are functionally dominant over p53 mutants. The novel nanomicelles restore a wild-type phenotype, and have very low toxicity to noncancerous cells. These properties of the novel compositions overcome many of the problems prior approaches have encountered in attempting to treat p53-mutant cancer cells. The novel Cer nanomicelles may be used in treating p53-associated cancers.

Although short-carbon-chain Cers, such as exogenous and cell-permeable C₆-Cer, can modulate apoptosis and gene regulation, longer-chain Cers, such as C₁₈-Cer, are expected to have greater potency. Using MBO-asGCS to suppress GCS restored wild-type p53 expression and p53-dependent apoptosis, and increased the levels of C₁₈-Cer detected by ESI-MS/MS in p53-mutant cancer cells.

More generally, we have discovered that restoring the expression of wild-type (wt) p53 protein in cancer cells that are heterozygous for wild-type p53 and mutant p53 not only suppresses the p53 mutant's oncogenic functions, it also suppresses tumor growth. We have also discovered a method for restoring expression of wild-type (wt) p53 protein in cancer cells that are heterozygous for wild-type p53 and mutant p53.

The novel compositions may be administered as a sole therapy, or co-administered with other anti-cancer therapies known in the art, such as doxorubicin.

Without wishing to be bound by this hypothesis, our underlying rationale is that ceramide (Cer) modulates post-transcriptional processes to restore wt p53 protein expression in heterozygous mutant cancer cells. We demonstrate the restorative effects of Cer on wt p53 expression and functions in cell culture in vitro, and then in animal studies in mice carrying missense p53 mutant tumors, before conducting clinical trials in human patients, all in accordance with applicable laws and regulations.

The invention is based on our improved understanding of how expression of the wt protein is regulated post-transcriptionally in heterozygous p53-mutant cells. The invention allows malignant progression to be inhibited or halted in cancers associated with p53 mutations. The novel treatment may be used alone or in conjunction with other treatments.

Water-soluble nanomicelles, which are effectively solubilized ceramides), are used as therapeutic agents to target p53-associated cancers. Restoring wt p53 expression and function in cells with p53 mutants inhibits growth and metastasis of cancers, and promotes apoptosis of p53-mutant cancer cells.

To the inventors' knowledge, no one has previously reported modulating wild-type p53 expression to restore wt p53 function in p53-mutant cancer cells.

FIG. 1 illustrates schematically the pathways of certain embodiments of the invention. Some abbreviations used in FIG. 1: GCS=glucosylceramide synthase. HSP-70=heat shock protein 70. bFGF=basic fibroblast growth factor. MDR1=multidrug resistance gene 1. TERT=telomerase reverse transcriptase. DRAM=damage-regulated autophagy modulator.

Ceramides are a family of closely-related molecules. Over 200 of structurally distinct mammalian ceramides have been reported. Below is a generic ceramide structure:

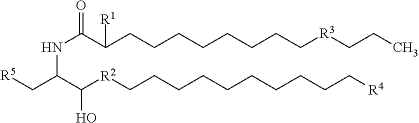

wherein:
R¹ is —H or —OH;
R² is —(CH₂)₃— or —CH═CH—CH₂— or —CH(OH)—CH—CH₂— or —CH═CH—CH(OH)—;
R³ is —(CH₂)ₙ—; where n=0, 1, 2, 3, 4, 5, 6, 7, 8, 9, 10, or 11;
R⁴ is —CH₃, —CH₂—CH₃, or CH₂—CH₂—CH₃; and
R⁵ is —H or —OH; and
   wherein any chiral center in the molecule may have the R or S configuration, or an equal or unequal mixture of the two configurations; and wherein any carbon-carbon double bond in the molecule may have the E or Z configuration, or an equal or unequal mixture of the two configurations.

The biosynthesis of a Cer proceeds through a de novo pathway requiring (and regulated via) a Cer synthase (one or more of CerS1-6), or it proceeds through sphingomyelin degradation catalyzed by sphingomyelinase (SMase). Cer glycosylation, effected by glucosylceramide synthase (GCS), converts Cer to glucosylceramide, which is the major precursor of glycosphingolipids.

The most effective Cers for therapeutic purposes are identified by comparisons carried out by transfection of CerS (CerS 1-6), or by silencing CerS expression with small interfering RNA (siRNA). Those with the most pronounced restorative impact on wt p53 expression and functional dominance over mutants in cells will be used for cancer therapy. Exogenous Cers (e.g., C₆-Cer, C₁₂₋₁₈-Cer, etc.) or modified Cers will be solubilized with rubusoside (RUB) to generate nanomicelles (Cer-RUB), and thereby substantially increase the water solubility of the ceramides. Cer-RUB nanomicelles are used to demonstrate the restorative effects of Cer in cell models and in vivo studies (animal and human). Human ovarian cancer cells NCI/ADR-RES (deletion mutation in Exon 5) and OVCAR-3 (missense R248G), isogenic human colon cancer cells SW48 TP53^{-/+} (missense R248W), and mouse breast cancer SVTneg2 (missense G242A, corresponding to human hot-spot G245A) will be used to demonstrate the restorative effects of Cer.

Our preliminary data have shown that Cer modulates post-transcriptional processes. Without wishing to be bound by this hypothesis, we believe that post-transcriptional regulation affects pre-mRNA splicing to favor wt p53 mRNA over mutant mRNA, thereby bringing about predominant expression of wt p53 protein in heterozygous p53 cells.

We will further confirm the effects of Cer-RUB nanomicelles on wt p53 expression in animal models, including transgenic mice (129S4-Trp53^{tm21tyj}, p53R172H), nude mice bearing orthotopic breast cancer (by mouse SVTneg2 cells, missense G242A), and nude mice bearing patient-derived tumor xenografts (PDX, ovarian, colorectal cancer patients; missense in DBD).

We will also further study the pharmacokinetics of Cer-RUB nanomicelles in transgenic mice and in mice bearing orthotopic tumors. The therapeutic effects of Cer-RUB nanomicelles will be further confirmed in mice with p53-mutant PDX models. The p53 mutations activate p53-repressive genes (HSP-70, bFGF, MDR1, β-catenin, hTERT, CD44, mTOR) to promote tumor progression and drug resistance. The wt p53 function is restored by Cer. Without wishing to be bound by this hypothesis, we believe that the restoration involves activation of p53-responsive genes or p53-targeted genes (e.g., p21, Fas, Bax, Puma, DRAM, microRNA-34) to eradicate tumors by cell-cycle arrest, autophagy, and apoptosis.

### BRIEF DESCRIPTION OF THE DRAWINGS

FIG. 1 illustrates schematically the pathways of certain embodiments of the invention.

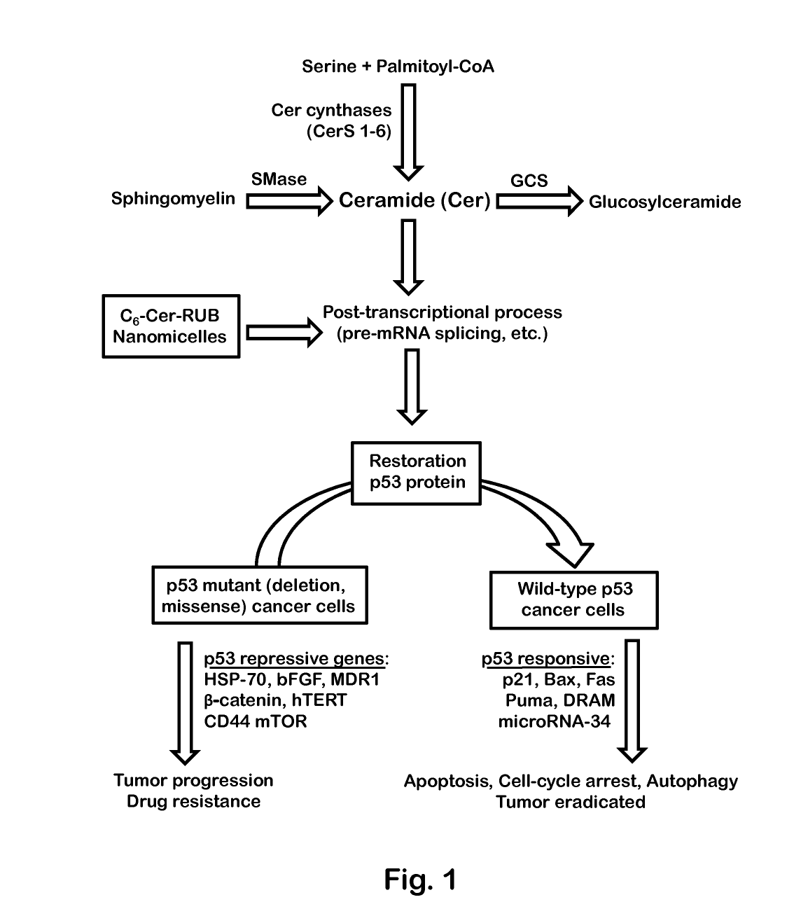

FIG. 2 depicts PP1 activity in NCI/ADR-RES cells with various treatments.

FIG. 3 depicts cell viability (as a percentage of control) after treatment with varying concentrations of C6-Ceramide.

FIG. 4 depicts cell viability (as a percentage of control) after treatment with varying concentrations of C6-Ceramide-Rubusoside.

FIG. 5 depicts cell viability (as a percentage of control) after treatment with varying concentrations of Rubusoside.

FIG. 6 depicts tumor volume as a function of time for treatment with doxorubicin alone, or treatment with co-administration of doxorubicin and ceramide-RUB nanomicelles.

FIG. 7 depicts tumor weight for treatment with doxorubicin alone, or co-administration of doxorubicin and ceramide-RUB nanomicelles, at the conclusion of the 30-day trial.

### MODES FOR CARRYING OUT THE INVENTION

### EXAMPLE 1

The Role of Cer in Restoring wt p53 Expression in Cancer Cells Harboring p53 Mutants.

A series of experiments is conducted to better understand the mechanism by which Cer restores wt p53 expression in cells with various p53 mutants, and to identify the Cer species that are most effective in restoring wt p53 expression. Without wishing to be bound by this hypothesis, we hypothesize that Cer modulates the restoration of p53 protein expression via post-transcriptional processes involving p53 mRNA splicing. Experiments and testing include the generation of Cer-RUB nanomicelles, genetic manipulation of cellular Cer synthesis, better characterization of Cer restorative effects in cancer cells harboring various p53 mutants (e.g., deletion, missense), and comparing effects of different Cer species.

### EXAMPLE 2

Cer-RUB Nanomicelles Enhance Cer Water Solubility.

We solubilized Cers by complexation with rubusoside (RUB). Compared to native C₆-Cer, the aqueous solubility of C₆-Cer-RUB was enhanced more than 2,000-fold (2 mg/ml vs. <1.0 μg/ml). The C₆-Cer-RUB complex in water solution was mono-disperse as measured by dynamic light scattering (Nanotrac 251), with an average diameter of ˜4 nm, and a distribution of diameters ranging from ˜2.0 nm to ˜6.5 nm. The C₆-Cer-RUB complex was readily dried into a powder form, which we have observed to be stable at room temperature for at least several weeks, if not longer. The C₆-Cer-RUB complex in aqueous solution is colorless, even at a concentration of 2 mg/mL. (A less pure source of rubusoside might result in a greenish or brownish tinge to the solution.) Alternatively, nanomicelles could be formed by mixing the components in water, for example by homogenization at 12,000 rpm for 10 minutes.

To test water-solubility, different amounts of C₆-Cer and RUB were mixed at a 1:10 ratio (w/w) in ethanol to prepare the C₆-Cer-RUB complex. The ethanol solvent was then removed to leave a white powder. The powder was stored at 4° C. until used.

C₆-Cer-RUB (powder) and C₆-Cer (non-micellar, without rubusoside) were reconstituted in RPMI-1640 medium at concentrations of 2 mg/ml and 0.1 μg/ml, respectively.

The aqueous solution of C₆-Cer-RUB was separated on an Alltech Prevail C18 column (250 mm×4.6 mm i.d.; 5 μm) with an elution program gradient (0-15 min, 60 to 90% methanol with 0.1% phosphoric acid; 1.0 ml/min). Output was observed by measuring absorbance at 215 nm. C₆-Cer and RUB eluted at 3.6 min and 19.3 min, respectively. (data not shown).

Using the novel nanomicelles, the total concentration of ceramide in aqueous solution can be two times, three times, four times, five times, ten times, or even more than the aqueous solubility of otherwise identical ceramide would be in the absence of a steviol glycoside solubilizer.

### EXAMPLE 3

The Restorative Effects of Cer on wt p53 Expression and Function in Mice Carrying Missense p53 Mutant Tumors.

Long-carbon-chain Cers, such as C₁₈-Cer, are expected to have greater potency than short-chain Cers. However, their hydrophobic nature has previously limited their use. Our novel, water-soluble Cer-RUB complexes overcome these problems, and may be used to inhibit p53-mutant cancers in vivo. We will also examine the pharmacokinetics and pharmacodynamics of the novel water-soluble Cer nanomicelles in mice with p53-mutant tumors.

Because they are hydrophobic, little has previously been reported concerning the possible effects of exogenously supplied Cers, particularly long-carbon-chain Cers in vivo. In practicing the present invention, different effects on cell signaling and modulation of p53 gene expression can be obtained using Cers of different chain lengths, or mixtures of different chain lengths.

We examined the effects of suppressing GCS on wt p53 expression in tumor-bearing mice. In preliminary studies we found that MBO-asGCS restores p53 expression in p53 mutant xenografts. Athymic mice bearing NCI/ADR-RES tumors were treated for 32 days with doxorubicin alone (Dox, 2 mg/kg once a week), Dox in combination with MBO-asGCS (1 mg/kg every 3 days), or Dox in combination with MBO-SC (scrambled control). Mixed-backbone oligonucleotides against GCS (MBO-asGCS) combined with doxorubicin (Dox) decreased tumor volume by 58% (157 vs. 376 mm³, p<0. 01), as compared with MBO-SC (scrambled control) combined with Dox, or as compared with Dox alone (data not shown).

Effects on tumor proteins were compared by Western blotting. Equal amounts of tumor proteins (100 ng/lane) were resolved by 4-20% PAGE and immunoblotted with antibodies. Silencing of GCS appeared to upregulate p53 and p53-responsive genes in tumors (p<0.001) (data not shown). MBO-asGCS treatments increased wt p53 by greater than 4-fold (p<0.001), as compared to saline or MBO-SC. The enhanced p53 levels, in turn, substantially upregulated the expression of p21^{Wafl/Cip1}, Bax, and Puma (data not shown).

Immunostaining confirmed these findings. Green fluorescence (FL), GCS-Alexa Fluor® 488-conjugated antibodies; red FL Fluor® 488-conjugated antibodies, and pp53-Alexa Fluor® 555-conjugated antibodies were used for staining. Nuclei were counterstained with DAPI. In TUNEL staining, apoptotic cells (TUNEL⁺) exhibited green fluorescence at ×200 magnification. Under fluorescence microscopy, MBO-asGCS, but not MBO-SC, produced a significant increase in nuclear pp53 in tumors, while decreasing GCS protein in cytoplasm. TUNEL analysis indicated that approximately 80% of tumor cells treated with the combination of MBO-asGCS and Dox underwent apoptosis, compared to only 3 to 9% of apoptotic cells treated with Dox alone or treated with Dox combined with MBO-SC (data not shown).

### EXAMPLE 4

Minimal Rubusoside Toxicity.

Preliminary toxicity studies in normal mice found that up to 4 g rubusoside daily per kg body mass, administered orally for two weeks, did not cause any clinical signs of toxicity. Rubusoside (RUB) is a steviol glycoside. Steviol glycosides, extracted from the leaves of various plants, are often used as natural food sweeteners. The Accepted Daily Intake (ADI, mg/kg/day) of steviol glycosides is 4 mg/kg/day for steviol, which would be roughly comparable to 8-12 mg/kg/day for rubusoside. Rubusoside is generally recognized as safe (GRAS) as a food additive for the general public according to the World Health Organization (WHO) (2005). A WHO expert panel has set 760 mg/kg/day RUB as the no-observed-effect level (NOEL) (2005). Thus there is ample tolerance for avoiding toxicity when rubusoside is employed in the present invention.

### EXAMPLE 5

Cer Restores p53 Expression in Transgenic Mice.

We examined the effects of Cer on restoring wt p53 expression in p53 missense transgenic mice. The experimental animals were heterozygous 129S4-Trp53^{tm2.1Tyj} transgenic mice, which carry the mutation p53^{R172H/+}, and have a genetic predisposition to cancer. (Doyle et al., 2010; Olive et al., 2004; Yu et al., 2012) These mice were generously provided by the NCI Mouse Repository (Rockville, Md.). We analyzed the genotypes of offspring by PCR analysis of DNA from blood collected from the tail vein. Heterozygous (HTZ) p53^{R172H/+} mice were treated with C₆-Cer or C₆-Cer-RUB (1 mg/kg, i.p., every 3 days for 6 days). We observed that either C₆-Cer or C₆-Cer-RUB significantly increased protein levels of p53 and Bax in the small intestine, as compared to saline treatment (p<0.001) (data not shown). Interestingly, C₆-Cer-RUB, but not C₆-Cer, restored p21 expression in the small intestine of heterozygous p53^{R172H/+} mice (data not shown). Immunohistochemistry microscopy also showed significantly enhanced p21 for C₆-Cer-RUB, but not C₆-Cer, in the cytoplasm of cells from the small intestine (data not shown).

### EXAMPLE 6

Mechanisms through which Cer Modulates Posttranscriptional Processes to Restore Wild-Type p53 Expression over p53 Mutants.

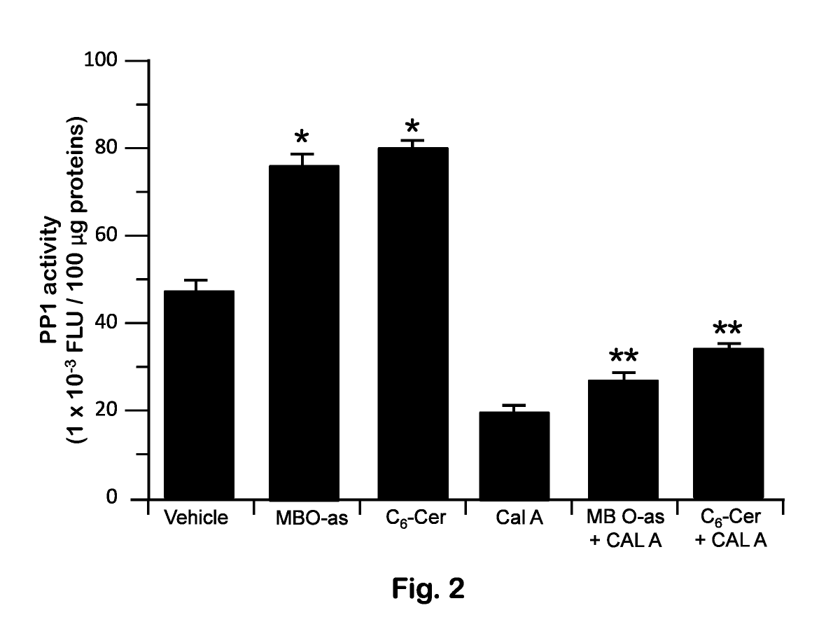

Cer can modulate gene expression, not only by activating transcription, but also by determining protein isoform. Our preliminary studies suggest that Cers modulate the binding specificity of the spliceosome to p53 RNA, leading to alternative splicing for the mature p53 mRNA. We assessed the roles of protein phosphatase 1 (PP1) and serine/arginine-rich splicing-factor 1 SRSF1 in Cer-induced wt p53 restoration via PP1 and SRSF1 in NCI/ADR-RES human ovarian cancer cells. We observed that MBO-asGCS and C₆-Cer significantly increased PP1 activity in NCI/ADR-RES cells. However, inhibition of PP1 by calyculin A (CalA) abolished these effects. Cells were pretreated with CalA (5 nM) for 4 hr, and then with either MBO-asGCS (MBO-as, 100 nM) or C₆-Cer (2.5 μM) for 72 hr. FIG. 2 depicts PP1 activity in NCI/ADR-RES cells with various treatments. MBO-asGCS treatments enhanced endogenous Cer levels, and thereby significantly increased SRSF1 levels in nuclear proteins (Western blotting, data not shown). These increases were correlated with increases in wt p53 mRNA (exon 5) and pp53 (as assessed by RT-PCR amplification, data not shown).

### EXAMPLE 7

Steviol Glycosides.

The initial embodiments of this invention have employed rubusoside. More generally, other steviol glycosides may be used in lieu of (or in addition to) rubusoside. Steviol glycosides include rubusoside, stevioside, rebaudioside A, rebaudioside B, rebaudioside C, rebaudioside D, rebaudioside E, dulcoside A.

### EXAMPLE 8

In Vitro Testing

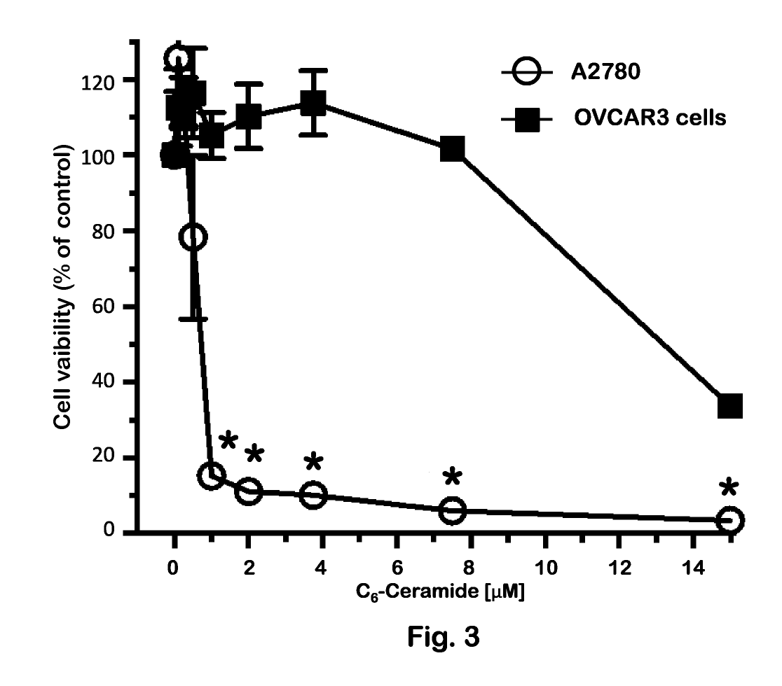

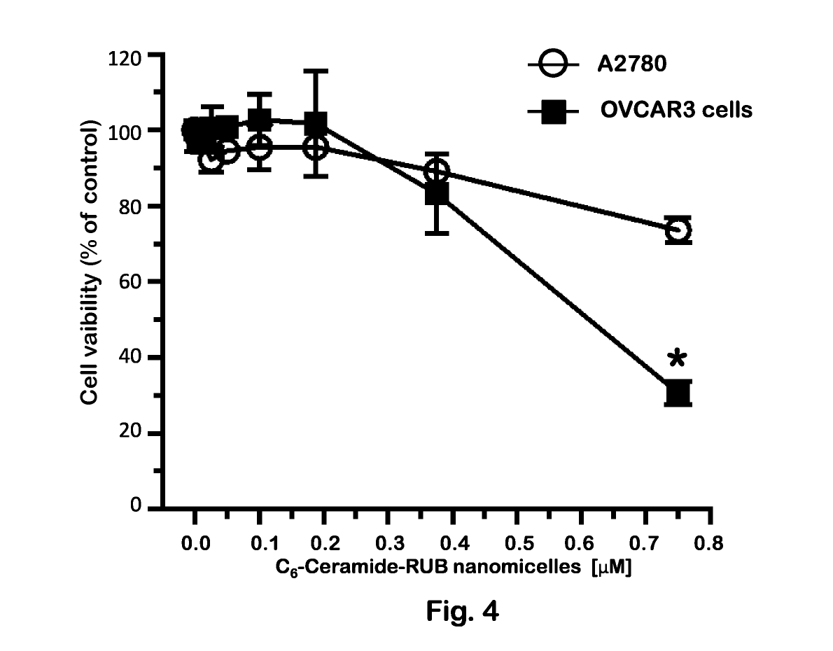

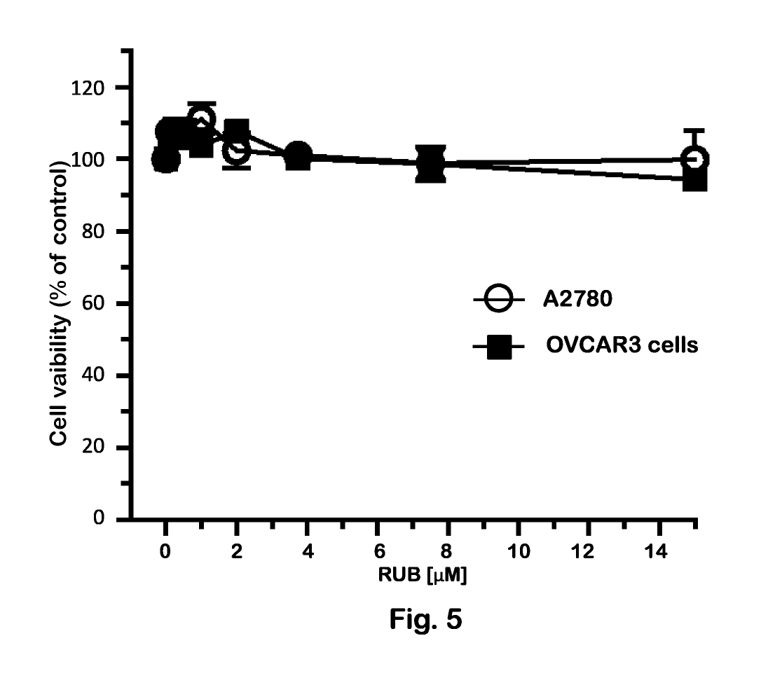

We examined the effect of Ceramide-RUB nanomicelles on p53 restoration in cancer cell models. Human OVCAR3 ovarian cancer cells carry a p53 R248 missense mutation, and are resistant to many anticancer drugs. OVCAR3 cells displayed 12 times more resistance to C6-Ceramide (IC50, 12.5 vs. 0.9 μM) than did A2780 ovarian cancer cells (which are p53 wild-type). However, Ceramide-RUB nanomicelles increased C6-ceramide effects on killing cancer cells more than 20 times (IC50, 0.6 vs. 12.5 μM) in OVCAR3 cells. Rubusoside itself showed no cytotoxicity towards either A2780 or OVCAR-3 cells at any concentration tested up to 15 μM. FIG. 3 depicts cell viability (as a percentage of control) after treatment for 72 hours with varying concentrations of C6-Ceramide. FIG. 4 depicts cell viability (as a percentage of control) after treatment for 72 hours with varying concentrations of C6-Ceramide-Rubusoside. FIG. 5 depicts cell viability (as a percentage of control) after treatment for 72 hours with varying concentrations of Rubusoside.

### EXAMPLE 9

In Vivo Testing

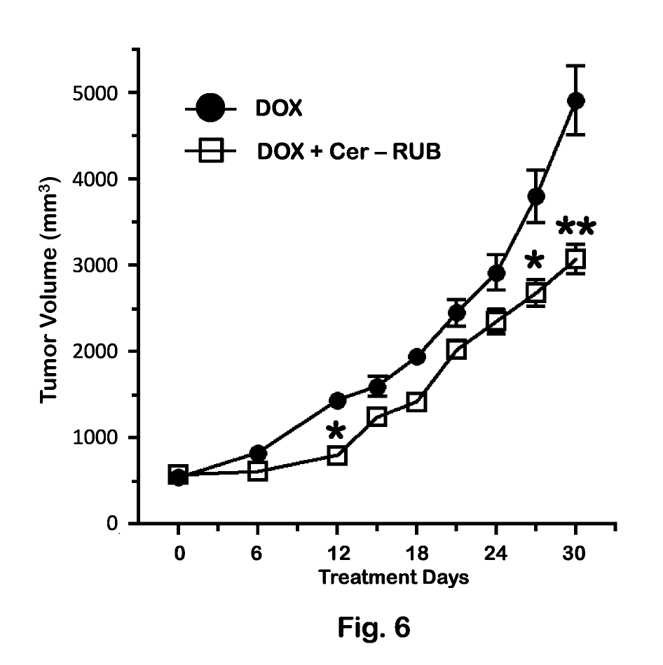

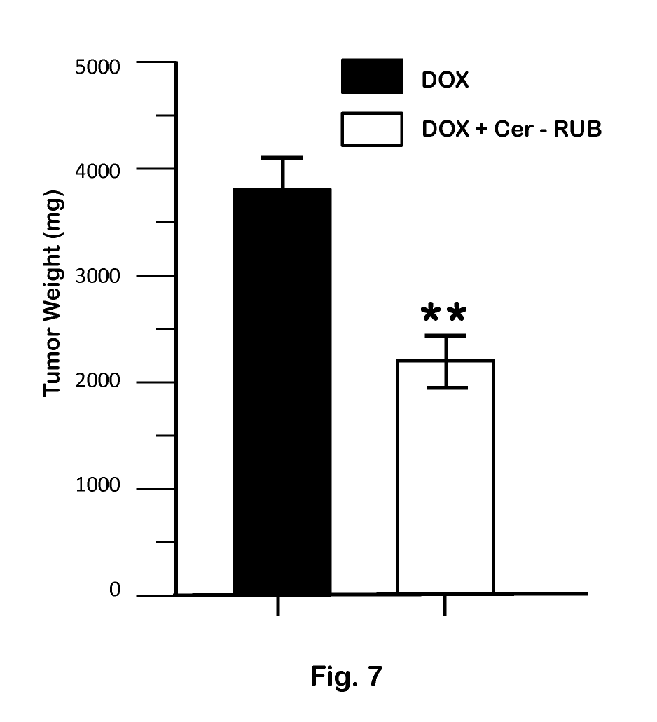

We next examined the therapeutic effect of Ceramide-RUB nanomicelles in vivo in p53-missense-mutant, tumor-bearing mice. We co-administered doxorubicin with ceramide-RUB nanomicelles (Cer-RUB, 4 mg/kg, equivalent to 0.1 mg/kg of C6-ceramide; intraperitoneal administration, every 3 days for 30 days) to treat xenograft advanced tumors that had been generated by inoculation of isogenic human colon cancer SW48 TP53^{-/+} (missense R248W) cells (2×10⁷ cells/mouse). Doxorubicin, whether administered alone or as co-therapy, was administered 0.2 mg/kg by intraperitoneal injection, every 6 days for 30 days. The tumors were allowed to grow more than one month before treatment began, to tumor volumes >500 mm³. The ceramide-RUB nanomicelles significantly enhanced the therapeutic effect of doxorubicin. The combination of Ceramide-RUB with doxorubicin significantly decreased the tumor volume and weight to ˜50% of that seen for doxorubicin alone. FIG. 6 depicts tumor volume as a function of time for treatment with doxorubicin alone, or treatment with co-administration of doxorubicin and ceramide-RUB nanomicelles. FIG. 7 depicts tumor weight for treatment with doxorubicin alone, or co-administration of doxorubicin and ceramide-RUB nanomicelles, at the conclusion of the 30-day trial. (To date, resource limitations have not yet permitted us to test the Ceramide-RUB nanomicelles as a monotherapy in vivo, although we plan to conduct such tests in the future.)

The complete disclosures of all references cited throughout this specification are hereby incorporated by reference, as are the complete disclosures of priority application U.S. 61/989,709, filed May 7, 2014, and of (Patwardhan et al. 2014). In the event of an otherwise irreconcilable conflict, however, the present specification shall control.

Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like. Further, a “functional group” or a “group” may consist of just one atom, or it may contain several atoms.

Ranges can be expressed herein as from “about” one particular value, to “about” another particular value. When such a range is expressed, another aspect includes from the one particular value to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight (or mass) of a particular element or component in a composition denotes the weight (or mass) relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight (or mass) of the formulation or composition in which the component is included.

As used herein, the terms “optional” or “optionally” means that the subsequently described event or circumstance may or may not occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses and embryos, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target histamine receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. The permissible substituents can be one or more, and they can be the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. It is also contemplated that, in certain aspects, unless expressly indicated to the contrary, individual substituents can be further optionally substituted (i.e., further substituted or unsubstituted).

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and each mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these compounds, called stereoisomers, are identical except that (at each chiral center) they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,R) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Unless otherwise expressly stated, it is in no way intended that any method set forth herein be construed as requiring that its steps be performed in any specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification. Those of skill in the art will recognize that, in some instances, it is implicit that at least some of the steps of a synthesis or of a separation should be carried out in a particular order to produce the desired result.

The compositions used in the present invention may be administered to a patient by any suitable means, including intravenous, parenteral, subcutaneous, intrapulmonary, and intranasal administration. Parenteral infusions include intramuscular, intravenous, intraarterial, or intraperitoneal administration. The compounds may also be administered transdermally, for example in the form of a slow-release subcutaneous implant. They may also be administered by inhalation.

Pharmaceutically acceptable carrier preparations include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampoule, each containing a unit dose amount, or in the form of a container containing multiple doses.

Compounds and compositions in accordance with the present invention may be formulated into therapeutic compositions as pharmaceutically acceptable salts. These salts include acid addition salts formed with inorganic acids, for example hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

A method for controlling the duration of action comprises incorporating an active compound or composition into particles of a polymeric substance such as a polyester, peptide, hydrogel, polylactide/glycolide copolymer, or ethylenevinylacetate copolymers. Alternatively, an active compound may be encapsulated in nanoparticles or microcapsules by techniques otherwise known in the art including, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

As used herein, an “effective amount” of a compound or composition is an amount, that when administered to a patient (whether as a single dose or as a time course of treatment) achieves one or more of the following outcomes to a clinically significant degree; or alternatively, to a statistically significant degree as compared to control: (1) increasing muscle mass, or (2) increasing the formation of thermogenic brown adipose tissue (BAT), or (3) decreasing white adipose tissue (WAT). “Statistical significance” means significance at the P<0.05 level, or such other measure of statistical significance as would be used by those of skill in the art of biomedical statistics in the context of a particular type of treatment or prophylaxis.

A specific “effective amount” for any particular patient will depend upon a variety of factors including the disorder being treated and the severity of the disorder; the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts. For example, it is well within the skill of the art to start doses of a compound at levels lower than those required to achieve the desired therapeutic effect and to gradually increase the dosage until the desired effect is achieved. If desired, the effective dose can be divided into multiple doses for purposes of administration. Consequently, single dose compositions can contain such amounts or submultiples thereof to make up the full dose. The dosage can be adjusted by the individual physician in the event of any contraindications. Dosage can vary, and can be administered in one or more dose administrations as needed.

Compositions of the present invention may be administered as a monotherapy for treatment of cancers, or co-administered with other therapeutic agents known in the art for treating cancers

The present invention may be used in vertebrates generally, including mammals such as humans, dogs, cats, horses, pigs, and cattle.

### REFERENCES

1. Evaluation of certain food additives. World Health Organ Tech Rep Ser 928, 1-156 (2005).
2. Adiseshaiah, P. P., Clogston, J. D., McLeland, C. B., Rodriguez, J., Potter, T. M., Neun, B. W., Skoczen, S. L., Shanmugavelandy, S. S., Kester, M., Stern, S. T., and McNeil, S. E. (2013). Synergistic combination therapy with nanoliposomal C6-ceramide and vinblastine is associated with autophagy dysfunction in hepatocarcinoma and colorectal cancer models. Cancer Lett 337, 254-265.
3. Bielawski, J., Pierce, J. S., Snider, J., Rembiesa, B., Szulc, Z. M., and Bielawska, A. (2009). Comprehensive quantitative analysis of bioactive sphingolipids by high-performance liquid chromatography-tandem mass spectrometry. Methods Mol Biol 579, 443-467.
4. Bielawski, J., Szulc, Z. M., Hannun, Y A., and Bielawska, A. (2006). Simultaneous quantitative analysis of bioactive sphingolipids by high-performance liquid chromatography-tandem mass spectrometry. Methods 39, 82-91.
5. Blons, H., and Laurent-Puig, P. (2003). TP53 and head and neck neoplasms. Hum Mutat 21, 252-257.
6. Bose, R., Verheij, M., Haimovitz-Friedman, A., Scotto, K., Fuks, Z., and Kolesnick, R. (1995). Ceramide synthase mediates daunorubicin-induced apoptosis: an alternative mechanism for generating death signals. Cell 82, 405-414.
7. Brosh, R., and Rotter, V. (2009). When mutants gain new powers: news from the mutant p53 field. Nat Rev Cancer 9, 701-713.
8. Bruno, A. P., Laurent, G, Averbeck, D., Demur, C., Bonnet, J., Bettaieb, A., Levade, T., and Jaffrezou, J. P. (1998). Lack of ceramide generation in TF-1 human myeloid leukemic cells resistant to ionizing radiation. Cell Death Differ 5, 172-182.
9. Bullock, A. N., and Fersht, A. R. (2001). Rescuing the function of mutant p53. Nat Rev Cancer 1, 68-76.
10. Chalfant, C. E., Kishikawa, K., Mumby, M. C., Kamibayashi, C., Bielawska, A., and Hannun, Y A. (1999). Long chain ceramides activate protein phosphatase-1 and protein phosphatase-2A. Activation is stereospecific and regulated by phosphatidic acid. J Biol Chem 274, 20313-20317.
11. Chalfant, C. E., Rathman, K., Pinkerman, R. L., Wood, R. E., Obeid, L. M., Ogretmen, B., and Hannun, Y A. (2002). De novo ceramide regulates the alternative splicing of caspase 9 and Bcl-x in A549 lung adenocarcinoma cells. Dependence on protein phosphatase-1. J Biol Chem 277, 12587-12595.
12. Chen, F., Wang, W., and El-Deiry, W. S. (2010). Current strategies to target p53 in cancer. Biochem Pharmacol 80, 724-730.
13. Cheng, J. C., Bai, A., Beckham, T. H., Marrison, S. T., Yount, C. L., Young, K., Lu, P., Bartlett, A. M., Wu, B. X., Keane, B. J., et al. (2013). Radiation-induced acid ceramidase confers prostate cancer resistance and tumor relapse. J Clin Invest 123, 4344-4358.
14. Cheok, C. F., and Lane, D. P. (2012). Seeking synergy in p53 transcriptional activation for cancer therapy. Discov Med 14, 263-271.
15. Doyle, B., Morton, J. P., Delaney, D. W., Ridgway, R. A., Wilkins, J. A., and Sansom, O. J. (2010). p53 mutation and loss have different effects on tumourigenesis in a novel mouse model of pleomorphic rhabdomyosarcoma. J Pathol 222, 129-137.
16. Elojeimy, S., Liu, X., McKillop, J. C., El-Zawahry, A. M., Holman, D. H., Cheng, J. Y, Meacham, W. D., Mandy, A. E., Saad, A. F., Turner, L. S., et al. (2007). Role of acid ceramidase in resistance to FasL: therapeutic approaches based on acid ceramidase inhibitors and FasL gene therapy. Mol Ther 15, 1259-1263.
17. Giussani, P., Bassi, R., Anelli, V., Brioschi, L., De Zen, F., Riccitelli, E., Caroli, M., Campanella, R., Gaini, S. M., Viani, P., and Riboni, L. (2012). Glucosylceramide synthase protects glioblastoma cells against autophagic and apoptotic death induced by temozolomide and Paclitaxel. Cancer Invest 30, 27-37.
18. Gomez-Munoz, A., Martin, A., O'Brien, L., and Brindley, D. N. (1994). Cell-permeable ceramides inhibit the stimulation of DNA synthesis and phospholipase D activity by phosphatidate and lysophosphatidate in rat fibroblasts. J Biol Chem 269, 8937-8943.
19. Gottschalk, B., and Klein, A. (2013). Restoration of wild-type p53 in drug-resistant mouse breast cancer cells leads to differential gene expression, but is not sufficient to overcome the malignant phenotype. Mol Cell Biochem 379, 213-227.
20. Grosch, S., Schiffmann, S., and Geisslinger, G (2012). Chain length-specific properties of ceramides. Prog Lipid Res 51, 50-62.
21. Hanel, W., Marchenko, N., Xu, S., Yu, S. X., Weng, W., and Moll, U. (2013). Two hot spot mutant p53 mouse models display differential gain of function in tumorigenesis. Cell Death Differ 20, 898-909.
22. Hannun, Y A., and Obeid, L. M. (2008). Principles of bioactive lipid signalling: lessons from sphingolipids. Nat Rev Mol Cell Biol 9, 139-150.
23. Hannun, Y. A., and Obeid, L. M. (2011). Many ceramides. J Biol Chem 286, 27855-27862.
24. Hermeking, H. (2007). p53 enters the microRNA world. Cancer Cell 12, 414-418.
25. Hollstein, M., Sidransky, D., Vogelstein, B., and Harris, C. C. (1991). p53 mutations in human cancers. Science 253, 49-53.
26. Iacopetta, B. (2003). TP53 mutation in colorectal cancer. Hum Mutat 21, 271-276.
27. Issaeva, N., Bozko, P., Enge, M., Protopopova, M., Verhoef, L. G, Masucci, M., Pramanik, A., and Selivanova, G (2004). Small molecule RITA binds to p53, blocks p53-HDM-2 interaction and activates p53 function in tumors. Nat Med 10, 1321-1328.
28. Jiang, Y, DiVittore, N. A., Kaiser, J. M., Shanmugavelandy, S. S., Fritz, J. L., Heakal, Y, Tagaram, H. R., Cheng, H., Cabot, M. C., Staveley-O'Carroll, K. F., et al. (2011). Combinatorial therapies improve the therapeutic efficacy of nanoliposomal ceramide for pancreatic cancer. Cancer Biol Ther 12, 574-585.
29. Kato, S., Han, S. Y, Liu, W., Otsuka, K., Shibata, H., Kanamaru, R., and Ishioka, C. (2003). Understanding the function-structure and function-mutation relationships of p53 tumor suppressor protein by high-resolution missense mutation analysis. Proc Natl Acad Sci U S A 100, 8424-8429.
30. Kenzelmann Broz, D., Spano Mello, S., Bieging, K. T., Jiang, D., Dusek, R. L., Brady, C. A., Sidow, A., and Attardi, L. D. (2013). Global genomic profiling reveals an extensive p53-regulated autophagy program contributing to key p53 responses. Genes Dev 27, 1016-1031.
31. Kroesen, B. J., Pettus, B., Luberto, C., Busman, M., Sietsma, H., de Leij, L., and Hannun, Y A. (2001). Induction of apoptosis through B-cell receptor cross-linking occurs via de novo generated C16-ceramide and involves mitochondria. J Biol Chem 276,13606-13614.
32. Lang, G. A., Iwakuma, T., Suh, Y A., Liu, G, Rao, V. A., Parant, J. M., Valentin-Vega, Y A., Terzian, T., Caldwell, L. C., Strong, L. C., et al. (2004). Gain of function of a p53 hot spot mutation in a mouse model of Li-Fraumeni syndrome. Cell 119, 861-872.
33. Levy, M., and Futerman, A. H. (2010). Mammalian ceramide synthases. IUBMB Life 62, 347-356.
34. Liu, J., Ma, Q., Zhang, M., Wang, X., Zhang, D., Li, W., Wang, F., and Wu, E. (2012). Alterations of TP53 are associated with a poor outcome for patients with hepatocellular carcinoma: evidence from a systematic review and meta-analysis. Eur J Cancer 48, 2328-2338.
35. Liu, Y. Y. (2011). Resuscitating wild-type p53 expression by disrupting ceramide glycosylation: a novel approach to target mutant p53 tumors. Cancer Res 71, 6295-6299.
36. Liu, Y Y, Han, T. Y, Giuliano, A. E., and Cabot, M. C. (2001). Ceramide glycosylation potentiates cellular multidrug resistance. Faseb J 15, 719-730.
37. Liu, Y Y, Hill, R. A., and Li, Y T. (2013). Ceramide glycosylation catalyzed by glucosylceramide synthase and cancer drug resistance. Adv Cancer Res 117, 59-89.
38. Liu, Y Y, Patwardhan, G A., Bhinge, K., Gupta, V., Gu, X., and Jazwinski, S. M. (2011). Suppression of glucosylceramide synthase restores p53-dependent apoptosis in mutant p53 cancer cells. Cancer Res 71, 2276-2285.
39. Liu, Y Y, Yu, J. Y, Yin, D., Patwardhan, G A., Gupta, V., Hirabayashi, Y, Holleran, W. M., Giuliano, A. E., Jazwinski, S. M., Gouaze-Andersson, V., et al. (2008). A role for ceramide in driving cancer cell resistance to doxorubicin. Faseb J 22, 2541-2551.
40. Massiello, A., and Chalfant, C. E. (2006). SRp30a (ASF/SF2) regulates the alternative splicing of caspase-9 pre-mRNA and is required for ceramide-responsiveness. J Lipid Res 47, 892-897.
41. Meijer, A., Kruyt, F. A., van der Zee, A. G, Hollema, H., Le, P., Ten Hoor, K. A., Groothuis, G M., Quax, W. J., de Vries, E. G, and de Jong, S. (2013). Nutlin-3 preferentially sensitises wild-type p53-expressing cancer cells to DRS-selective TRAIL over rhTRAIL. Br J Cancer 109, 2685-2695.
42. Merrill, A. H., Jr. (2011). Sphingolipid and glycosphingolipid metabolic pathways in the era of sphingolipidomics. Chem Rev 111, 6387-6422.
43. Milner, J., and Medcalf, E. A. (1991). Cotranslation of activated mutant p53 with wild type drives the wild-type p53 protein into the mutant conformation. Cell 65, 765-774.
44. Mizutani, Y, Kihara, A., and Igarashi, Y (2005). Mammalian Lass6 and its related family members regulate synthesis of specific ceramides. Biochem J 390, 263-271.
45. Modrak, D. E., Leon, E., Goldenberg, D. M., and Gold, D. V. (2009). Ceramide regulates gemcitabine-induced senescence and apoptosis in human pancreatic cancer cell lines. Mol Cancer Res 7, 890-896.
46. Morad, S. A., and Cabot, M. C. (2013). Ceramide-orchestrated signalling in cancer cells. Nat Rev Cancer 13, 51-65.
47. Ogretmen, B., and Hannun, Y A. (2004). Biologically active sphingolipids in cancer pathogenesis and treatment. Nat Rev Cancer 4, 604-616.
48. Olive, K. P., Tuveson, D. A., Ruhe, Z. C., Yin, B., Willis, N. A., Bronson, R. T., Crowley, D., and Jacks, T. (2004). Mutant p53 gain of function in two mouse models of Li-Fraumeni syndrome. Cell 119, 847-860.
49. Olivier, M., Langerod, A., Carrieri, P., Bergh, J., Klaar, S., Eyfjord, J., Theillet, C., Rodriguez, C., Lidereau, R., Bieche, I., et al. (2006). The clinical value of somatic TP53 gene mutations in 1,794 patients with breast cancer. Clin Cancer Res 12, 1157-1167.
50. Patwardhan, G A., Zhang, Q. J., Yin, D., Gupta, V., Bao, J., Senkal, C. E., Ogretmen, B., Cabot, M. C., Shah, G V., Sylvester, P. W., et al. (2009). A new mixed-backbone oligonucleotide against glucosylceramide synthase Sensitizes multidrug-resistant tumors to apoptosis. PLoS One 4, e6938.
51. Patwardhan, G A., Hosain, S. B., Liu, D. X., Khiste, S. K., Zhao, Y, Bielawski, J., Jazwinski, S. M., Liu, Y Y (2014). Ceramide modulates pre-mRNA splicing to restore the expression of wild-type tumor suppressor p53 in deletion-mutant cancer cells. Biochemica et Biophysica Acta 1841, 1571-1580.
52. Peller, S., and Rotter, V. (2003). TP53 in hematological cancer: low incidence of mutations with significant clinical relevance. Hum Mutat 21, 277-284.
53. Petitjean, A., Achatz, M. I., Borresen-Dale, A. L., Hainaut, P., and Olivier, M. (2007a). TP53 mutations in human cancers: functional selection and impact on cancer prognosis and outcomes. Oncogene 26, 2157-2165.
54. Petitjean, A., Mathe, E., Kato, S., Ishioka, C., Tavtigian, S. V., Hainaut, P., and Olivier, M. (2007b). Impact of mutant p53 functional properties on TP53 mutation patterns and tumor phenotype: lessons from recent developments in the IARC TP53 database. Hum Mutat 28, 622-629.
55. Riebeling, C., Allegood, J. C., Wang, E., Merrill, A. H., Jr., and Futerman, A. H. (2003). Two mammalian longevity assurance gene (LAG1) family members, trh1 and trh4, regulate dihydroceramide synthesis using different fatty acyl-CoA donors. J Biol Chem 278, 43452-43459.
56. Schuijer, M., and Berns, E. M. (2003). TP53 and ovarian cancer. Hum Mutat 21, 285-291.
57. Senkal, C. E., Ponnusamy, S., Rossi, M. J., Bialewski, J., Sinha, D., Jiang, J. C., Jazwinski, S. M., Hannun, Y A., and Ogretmen, B. (2007). Role of human longevity assurance gene 1 and C18-ceramide in chemotherapy-induced cell death in human head and neck squamous cell carcinomas. Mol Cancer Ther 6, 712-722.
58. Shangary, S., Qin, D., McEachern, D., Liu, M., Miller, R. S., Qiu, S., Nikolovska-Coleska, Z., Ding, K., Wang, G, Chen, J., et al. (2008). Temporal activation of p53 by a specific MDM2 inhibitor is selectively toxic to tumors and leads to complete tumor growth inhibition. Proc Natl Acad Sci U S A 105, 3933-3938.
59. Shi, J., and Zheng, D. (2009). An update on gene therapy in China. Curr Opin Mol Ther 11, 547-553.
60. Sigal, A., and Rotter, V. (2000). Oncogenic mutations of the p53 tumor suppressor: the demons of the guardian of the genome. Cancer Res 60, 6788-6793.
61. Skilling, J. S., Squatrito, R. C., Connor, J. P., Niemann, T., and Buller, R. E. (1996). p53 gene mutation analysis and antisense-mediated growth inhibition of human ovarian carcinoma cell lines. Gynecol Oncol 60, 72-80.
62. Soussi, T., and Beroud, C. (2001). Assessing TP53 status in human tumours to evaluate clinical outcome. Nat Rev Cancer 1, 233-240.
63. Stein, T., Crighton, D., Warnock, L. J., Milner, J., and White, R. J. (2002). Several regions of p53 are involved in repression of RNA polymerase III transcription. Oncogene 21, 5540-5547.
64. Stover, T. C., Sharma, A., Robertson, G P., and Kester, M. (2005). Systemic delivery of liposomal short-chain ceramide limits solid tumor growth in murine models of breast adenocarcinoma. Clin Cancer Res 11, 3465-3474.
65. Sur, S., Pagliarini, R., Bunz, F., Rago, C., Diaz, L. A., Jr., Kinzler, K. W., Vogelstein, B., and Papadopoulos, N. (2009). A panel of isogenic human cancer cells suggests a therapeutic approach for cancers with inactivated p53. Proc Natl Acad Sci U S A 106, 3964-3969.
66. Tang, H. Y, Zhao, K., Pizzolato, J. F., Fonarev, M., Langer, J. C., and Manfredi, J. J. (1998). Constitutive expression of the cyclin-dependent kinase inhibitor p21 is transcriptionally regulated by the tumor suppressor protein p53. J Biol Chem 273, 29156-29163.
67. Ueno, Y, Enomoto, T., Otsuki, Y, Sugita, N., Nakashima, R., Yoshino, K., Kuragaki, C., Ueda, Y, Aki, T., Ikegami, H., et al. (2006). Prognostic significance of p53 mutation in suboptimally resected advanced ovarian carcinoma treated with the combination chemotherapy of paclitaxel and carboplatin. Cancer Lett 241, 289-300.
68. van Oijen, M. G, and Slootweg, P. J. (2000). Gain-of-function mutations in the tumor suppressor gene p53. Clin Cancer Res 6, 2138-2145.
69. van Vlerken, L. E., Duan, Z., Little, S. R., Seiden, M. V., and Amiji, M. M. (2008). Biodistribution and pharmacokinetic analysis of Paclitaxel and ceramide administered in multifunctional polymer-blend nanoparticles in drug resistant breast cancer model. Mol Pharm 5, 516-526.
70. Vassilev, L. T., Vu, B. T., Graves, B., Carvajal, D., Podlaski, F., Filipovic, Z., Kong, N., Kammlott, U., Lukacs, C., Klein, C., et al. (2004). In vivo activation of the p53 pathway by small-molecule antagonists of MDM2. Science 303, 844-848.
71. Ventura, A., Kirsch, D. G, McLaughlin, M. E., Tuveson, D. A., Grimm, J., Lintault, L., Newman, J., Reczek, E. E., Weissleder, R., and Jacks, T. (2007). Restoration of p53 function leads to tumour regression in vivo. Nature 445, 661-665.
72. Vogelstein, B., Lane, D., and Levine, A. J. (2000). Surfing the p53 network. Nature 408, 307-310.
73. Vousden, K. H., and Prives, C. (2009). Blinded by the Light: The Growing Complexity of p53. Cell 137, 413-431.
74. Westra, J. L., Schaapveld, M., Hollema, H., de Boer, J. P., Kraak, M. M., de Jong, D., ter Elst, A., Mulder, N. H., Buys, C. H., Hofstra, R. M., and Plukker, J. T. (2005). Determination of TP53 mutation is more relevant than microsatellite instability status for the prediction of disease-free survival in adjuvant-treated stage III colon cancer patients. J Clin Oncol 23, 5635-5643.
75. Wooten-Blanks, L. G, Song, P., Senkal, C. E., and Ogretmen, B. (2007). Mechanisms of ceramide-mediated repression of the human telomerase reverse transcriptase promoter via deacetylation of Sp3 by histone deacetylase 1. FASEB J 21, 3386-3397.
76. Wooten, L. G, and Ogretmen, B. (2005). Spl/Sp3-dependent regulation of human telomerase reverse transcriptase promoter activity by the bioactive sphingolipid ceramide. J Biol Chem 280, 28867-28876.
77. Yaginuma, Y, and Westphal, H. (1992). Abnormal structure and expression of the p53 gene in human ovarian carcinoma cell lines. Cancer Res 52, 4196-4199.
78. Young, M. M., Kester, M., and Wang, H. G (2013). Sphingolipids: regulators of crosstalk between apoptosis and autophagy. J Lipid Res 54, 5-19.
79. Yu, X., Vazquez, A., Levine, A. J., and Carpizo, D. R. (2012). Allele-specific p53 mutant reactivation. Cancer Cell 21, 614-625.
80. Zeimet, A. G, and Marth, C. (2003). Why did p53 gene therapy fail in ovarian cancer? Lancet Oncol 4, 415-422.
81. Zhang, F., Koh, G Y, Hollingsworth, J., Russo, P. S., Stout, R. W., and Liu, Z. (2012). Reformulation of etoposide with solubility-enhancing rubusoside. Int J Pharm 434, 453-459.
82. Zhang, F., Koh, G Y, Jeansonne, D. P., Hollingsworth, J., Russo, P. S., Vicente, G, Stout, R. W., and Liu, Z. (2011). A novel solubility-enhanced curcumin formulation showing stability and maintenance of anticancer activity. J Pharm Sci 100, 2778-2789.

What is claimed:

1. A dry composition of matter comprising a complex of at least one steviol glycoside and at least one ceramide.

2. An aqueous solution of the composition of claim 1, wherein said steviol glycoside and said ceramide form micelles, and wherein the total concentration of ceramide in the aqueous solution is at least ten times greater than the aqueous solubility of otherwise identical ceramide would be in the absence of said steviol glycoside.

3. The aqueous solution of claim 2, wherein said micelles have diameters between about 2.0 nm and about 6.5 nm.

4. The composition of claim 1, wherein said at least one steviol glycoside comprises rubusoside.

5. The composition of claim 1, wherein said at least one steviol glycoside comprises stevioside.

6. The composition of claim 1, wherein said at least one steviol glycoside comprises rebaudioside A.

7. The composition of claim 1, wherein said at least one steviol glycoside is selected from the group consisting of rebaudioside B, rebaudioside C, rebaudioside D, rebaudioside E, dulcoside A.

8. The composition of claim 1, wherein said at least one ceramide is selected from the group consisting of C6 ceramide, C8 ceramide, C10 ceramide, and C12 ceramide.

9. A method for enhancing expression of wild-type p53 in cells that are heterozygous for wild-type TP53 and mutant TP53, said method comprising administering to the cells the composition of claim 1.

10. The method of claim 9, wherein said administration is carried out in vivo.

11. The method of claim 10, wherein said administration comprises consuming the composition orally.

12. The method of claim 10, wherein said administration comprises injecting an aqueous solution of the composition.

13. A method of inhibiting a cancer in a mammalian patient, wherein the cancer cells are heterozygous for wild-type TP53 and mutant TP53, said method comprising administering to the patient the composition of claim 1.

14. The method of claim 13, wherein said administration comprises consuming the composition orally.

15. The method of claim 13, wherein said administration comprises injecting an aqueous solution of the composition.

16. The method of claim 13, wherein said administration comprises topical administration to a skin cancer.

17. The method of claim 13, additionally comprising the co-administration to the patient of an anticancer therapeutic in addition to said composition.

18. The method of claim 17, wherein said anticancer therapeutic comprises doxorubicin.
